(19) United States
(12) Patent Application Publication
(10) Pub. No.: US 2012/0091218 A1
(43) Pub. Date: Apr. 19, 2012

(54) METHOD FOR THE PRODUCTION OF LOW VOC FRAGRANCED HYDROGEL SPHERES HAVING IMPROVED OPTICAL CLARITY

(75) Inventors: Tyler Mikkelsen, Scottsdale, AZ (US); Rosanne Benavides, Phoenix, AZ (US); Kevin Hafer, Chandler, AZ (US)

(73) Assignee: The Dial Corporation, Scottsdale, AZ (US)

(21) Appl. No.: 12/904,329

(22) Filed: Oct. 14, 2010

(51) Int. Cl. A61L 9/04

(52) U.S. Cl. 239/6; 512/4; 424/76.4

(57) ABSTRACT

A method for producing low-VOC fragranced hydrogel spheres of improved optical clarity and fragrance delivery is described and comprises the steps of combining a stable oil-in-water fragrance microemulsion absent co-solvents with a coarse dry granular, super-absorbent, hydrogel-forming copolymer. The resulting discrete hydrogel spheres have transparent optical beauty and liberate fragrance into an environment leaving behind only a small amount of emulsifier once evaporated. The air freshener produced by the disclosed method provides decoration in a home when the spheres are placed in a decorative jar.

### FIELD OF INVENTION

The present invention relates to passive air fresheners and in particular to a method for producing low-VOC fragranced hydrogel spheres of improved optical clarity and fragrance delivery by combining a stable oil-in-water fragrance microemulsion absent co-solvents with a coarse dry granular, super-absorbent, hydrogel-forming copolymer.

### BACKGROUND

Air fresheners have been in the marketplace and in the patent literature for decades. Consumer air fresheners run the gamut from static blocks of fragranced gel to programmable and playable electronic devices with heaters and/or fans, and correspondingly are sold across a wide price range. “Passive” refers to air fresheners that do not require any energy to operate. That is, a passive air freshener can scent a room by evaporating over time just sitting at ambient temperature in a home. These products evaporate to dryness over time, and their evaporation rate (and resultant product length of life) is usually determined by the volatility of the fragrance/solvent mixture and the surface area from which the volatile fragrance mixture can evaporate. Some passive air fresheners, particularly those with water-based liquid fragrance solutions, rely on cellulose emanating pads with large surface area, or large diameter fiber wicks, to evaporate the volatile liquid fragrance mixture into the room. Other typical passive air fresheners are static blocks of fragranced gel such as the Renuzit® Adjustables air freshener gel that has been in the market for many years. One problem with static gel air fresheners comprising a single contiguous block is that they surface dry and “skin over” in a short period of time, and the evaporation of volatiles then slows down considerably. Compounding this shortcoming is the fact that the surface area of a block of fragranced gel is small compared to the mass of the product. For example, a solid gelled air freshener product may weigh around 250 grams or more yet have only a surface area of around 150 cm², (assuming a container of fragranced gel measuring 10 cm×5 cm×5 cm). For such a product to be used up, about 250 grams of volatile material (minus the weight of the gelling agent, typically a small amount) must evaporate from only 150 cm² of surface area, and perhaps less than that if the fragranced gel is evaporated from the container in which it was cast. Another shortcoming of these solid fragranced products is that they are not very attractive. Although some transparent fragranced gels are known, and may be created using synthetic polymers such as polyacrylates or polyurethanes (e.g. as disclosed by Christenson, et. al in U.S. Pat. No. 6,960,625), the most cost effective way to make a low-VOC water-based air freshener gel is still to use a polysaccharide such as carrageen as the gelling agent and this necessarily results in an air freshener gel that is opaque. Additionally, many clear gel air fresheners using synthetic polymers incorporate high levels of co-solvent such as ethanol or isopropyl alcohol to ensure stability and clarity as the product dries out. New regulatory initiatives to improve air quality have centered on reduction of VOC's in many household and industrial products, and these regulations have affected, and will continue to affect, the formulation of consumer air fresheners. For example in California, gel air fresheners may be limited to less than 0.1% VOC excluding the fragrance volatiles. Such a restriction will undoubtedly require formulators to avoid solvents such as ethanol in air freshener gels.

Therefore what is still lacking in the industry is a passive gel air freshener product that is clear and attractive in appearance, with a larger surface area for evaporation, yet formulated to have low or no-VOC to be in compliance with changing regulations.

### SUMMARY OF THE INVENTION

It has now been discovered that hydrating coarse, super-absorbent polymer granules with a stable aqueous fragrance microemulsion that is free of co-solvent, results in low-VOC fragranced hydrogel spheres that have great fragrance delivery throughout their life and have incredible optical beauty. Unexpectedly, the spheres remain transparent as they dry up and shrink during use as an air freshener, even though there is no solvent to maintain stability as the spheres change in composition exposed to the room air over time. Adding to the decorative aspects of the present invention is use of a distribution of coarse particle size polymer granules to produce a pleasing distribution of swelled hydrogel sphere sizes. The key to producing fragranced hydrogel spheres of desirable optical beauty is to first form a stable, oil-in-water, nonionic fragrance microemulsion that is low in electrolytes and completely absent co-solvent, and use this microemulsion to hydrate the coarse super-absorbing polymer granules. The present inventive step is the discovery that low electrolyte levels through use of only nonionic emulsifiers in the fragrance microemulsion is essential to creating fragranced hydrogel spheres that have the desired optical transparency.

In one preferred embodiment of the present method, an polyacrylamide/sodium acrylate copolymer in the form of coarse dry granules ranging in particle size from about 1 mm to about 6 mm is combined with a stable oil-in-water microemulsion comprising a fragrance oil, at least one nonionic emulsifier, and water, resulting in low-VOC fragranced hydrogel spheres.

In another preferred embodiment of the present method, an polyacrylamide/potassium acrylate copolymer in the form of coarse dry granules ranging in particle size from about 1 mm to about 6 mm is combined with a stable oil-in-water microemulsion comprising a fragrance oil, at least one nonionic emulsifier, and water, resulting in low-VOC fragranced hydrogel spheres having greater than 20% light transmission at wavelengths above 400 nm, greater than 25% light transmission at wavelengths above 500 nm, and greater than 30% light transmission at wavelengths above 700 nm.

Such transparent, low-VOC fragranced hydrogel spheres produced by the present method give good fragrance delivery at ambient temperature and function well as passive decorative low-VOC air freshener compositions that maintain a pleasing appearance as the spheres dry up and shrink to residue

### BRIEF DESCRIPTION OF THE DRAWINGS

FIG. 1 is a plot of percent transmittance (% T) at 350-900 nm incident light for two different fragranced hydrogel spheres, showing the enhanced optical clarity of the fragranced hydrogel spheres possible from the present method and ingredients.

### DETAILED DESCRIPTION OF THE INVENTION

The following description is of exemplary embodiments only and is not intended to limit the scope, applicability or configuration of the invention in any way. Rather, the following description provides a convenient illustration for implementing exemplary embodiments of the invention. Various changes to the described embodiments may be made in the relative amounts of the ingredients and the conditions of the manufacturing method described without departing from the scope of the invention as set forth in the appended claims. Most importantly, changes in shape and size of the swelled fragrance spheres or changes to the size distribution of the spheres do not depart from the intended scope of the invention. Furthermore, changes to the order of addition of the ingredients, or changes to the temperatures and time of mixing do not depart from the intent of the invention. Although the present invention is described in utilitarian terms as a method to produce an air freshener comprising discrete fragranced gel spheres, other useful applications for the swelled polymer hydrogel spheres produced from the present method and having other volatile ingredients may be envisioned and fall within the scope of the present invention.

That said, the present invention relates to a method for producing a passive gel air freshener comprising discrete swelled spheres of fragranced gel that have desirable clarity and fragrance delivery. The fragranced hydrogel spheres produced by the present method are comprised of a super-absorbent polymer or copolymer (often abbreviated in the art as SAP) and an aqueous fragrance microemulsion. As will be described in detail below, the stability and clarity of the fragrance microemulsion predictably translates into the optical clarity of the resultant hydrogel spheres, and therefore it is desirable to first produce a stable aqueous fragrance microemulsion and then to combine the liquid with a particular dry granular SAP of specific granulometry in order to produce fragranced hydrogel spheres having the desirable optical transparency.

More specifically the present method of manufacture comprises the steps of (1) mixing all the ingredients minus the SAP together to form an aqueous fragrance microemulsion; and (2) then mixing the clear liquid microemulsion with the granular SAP to form the swelled spheres over a sufficient period of time to hydrate the granules. It is irrelevant if the fragrance microemulsion is poured on top of the dry SAP granules or if the SAP granules are dropped, sifted, etc. into the liquid. However, depending on the characteristics of the container in which the hydration of the SAP granules is to take place (e.g. if made of PET or other plastic), the SAP granules may tend to have static problems with the dry container if dropped therein, with the remedy being addition of the liquid into the container first and then addition of the SAP granules to the liquid filled container. Lastly, and as will be detailed below, the method of use of the air freshener spheres produced by the present method is to expose a number of the fragranced hydrogel spheres to an environment to be scented (e.g. a small room in a home), preferably by placing the spheres into a decorative, clear jar. The container mentioned above in the manufacturing process may of course be the same decorative, clear jar that the consumer places into their home.

VOC refers to “volatile organic compounds,” something that is highly regulated to reduce air pollution and global warming. By multiplying each of the solvent levels present in the individual raw materials by the amount of that raw material used in the composition, and then adding these fractional amounts, give the “VOC content” or “percent volatile organic compounds” in a composition. Ethanol, for example, may be a 100% VOC contributing raw material when it is “absolute alcohol,” whereas some raw materials, such as water or 100% active nonionic alcohol ethoxylate surfactants, may contribute no VOC at all to the composition. Frequently however, solvents such as ethanol or isopropyl alcohol are added to stabilize oil-in-water fragrance microemulsions, and using these solvents adds to the VOC content of the final composition. Removal of ethanol from the production of fragranced hydrogel spheres results in opaque spheres unless the fragrance microemulsion used to produce the spheres is created only with nonionic substances and only if the microemulsion has low electrolyte levels, and only when the SAP is properly chosen.

Herein, “transparency” is a term used qualitatively and only subjectively, and is meant to convey a characteristic seen by comparing undesirable, opaque hydrogel spheres with those produced by the present method/ingredients that appear by the naked eye to be “more transparent.” In other words, use of the terms “transparent,” “clarity,” “optical clarity,” or “optical beauty” herein, is only meant to convey that the product manufactured by the present method appears clear and not opaque when visually inspected. However, for the improvement in transparency in accordance with the present invention to be communicable, particular light transmittance percentage (% T) plots versus wavelengths of incident light for swelled, fragranced hydrogel spheres produced by the present method will be set out below. Surprisingly, the measured % T plots for the preferred spheres with optical beauty are considerably less than one would expect (e.g., never exceeding 35% T from 350-900 nm) considering the swelled hydrogel spheres produced herein look so optically appealing and much improved over alternative formulas and processes that produce opaque and cloudy air freshener spheres.

Super Absorbing Polymer

The polymer according to the present invention, to be mixed with the fragrance microemulsion, is preferably a super-absorbent material, and more specifically a super-absorbent polymer or SAP. These are materials that are capable of absorbing large amounts of water or aqueous solutions are thus referred to in the art as “hydrogel-forming” SAP's. In the context of the present invention, super-absorbents are synthetic organic polymers or copolymers that may be linear, branched, and optionally cross-linked. Preferred SAP's may contain acrylic acid, methacrylic acid, acrylamide, acrylic esters, and/or methacrylic esters as monomers, and may be homopolymers of any of the above-mentioned monomers. Alternative, the SAP's may be copolymers through combinations of acrylates, acrylamides, methacrylates, acrylic acid or methacrylic acid, or copolymers of any of these monomers with vinyl acetate, vinyl alcohol, maleic anhydride, or isobutylene-maleic anhydride. The SAP's may also be saponified graft polymers of acrylonitrile or graft polymers of starch and acrylic acid, polyvinyl alcohol, polyvinylpyrrolidone, polyvinyl alkyl ether, polyethylene oxide, polyacrylamide, and copolymers thereof, or their salts. In any case, such SAP's as those described above are capable of absorbing between about 50 and 200 times their own weight of water or hydrophilic solvent. The most common SAP's include the cross-linked sodium polyacrylate/polyacrylic acid polymers, at least for use in diapers where rapid absorption of liquids having high electrolyte content is required. Super-absorbents of this type are commercially available under the names Salsorb® (Ciba/Allied Colloids, Ltd.) and Cabloc® (Stockhausen, GmbH). Other SAP's that may find use in the present invention include, but are not limited to, the hydrogel-forming polymers set forth in U.S. Pat. Nos. 7,528,291; 7,504,551; 5,669,894; 5,559,335; 5,539,019; 5,250,642; 5,196,456; 5,145,906; 4,507,438; and, 4,295,987; and, in U.S. Pat. Application No. 2007/0185228, all incorporated herein by reference.

However, the most preferred super-absorbing polymers for use in the present invention are copolymers containing acrylamide and acrylic acid salt monomers. These copolymers may be alternating copolymers, random copolymers, block copolymers, or graft copolymers. They may be linear or branched, and optionally cross-linked. Particularly preferred polymers include: acrylamide/sodium acrylate cross-linked copolymer (CAS No. 25085-02-3) sold by Aekyung Specialty Chemicals Co, Ltd under the trade name Hisobead®, and by Stockhausen, GmbH under the trade name Praestol®, amongst other domestic and international suppliers; a copolymer of starch, with grafted side chains of copolymers of acrylamide and sodium acrylate, sold by Grain Processing Corporation under the trade name Water Lock® A-100; and, acrylamide/potassium acrylate cross-linked copolymer (CAS No. 31212-13-2) sold by Horticultural Alliance, Inc. under the trade name Horta-Sorb®, by Stockhausen, GmbH under the trade name StockSorb®, and by Novo-Tech, Inc. under the trade name Water Keep®, amongst other domestic and international suppliers. “CAS No.” refers to the identification number assigned by the Chemical Abstracts Service, which orchestrates a globally recognized identification system for chemical compounds and assigns unique identifiers to each and every chemical substance known. The CAS No. is particularly important when distinguishing between SAP's because these materials, often having abbreviated, creative trivial names and/or brand names, are easily confused. Without question the most preferred SAP herein is the cross-linked acrylamide/potassium acrylate copolymer identified by CAS No. 31212-13-2 because this particular SAP consistently produces clear fragranced hydrogel spheres provided that the granular SAP is combined with a solvent free, nonionic-based fragrance microemulsion. Use of the sodium salt (CAS 25085-02-3) gives a more opaque and less preferred hydrogel sphere.

The super-absorbent polymers/copolymers for use in the present invention, regardless of chemical composition, are preferably sourced in the form of a dry, coarse granulate with particle size range of from about 0.5 mm to about 6 mm, such that visibly large and discrete hydrogel spheres result upon admixture of the granulate with the aqueous microemulsion and after sufficient time for full absorption of liquid by the SAP. Granulates with larger granulometry (e.g., granules greater than 7 mm or so), or finely powdered SAP's, may be used to produce air fresheners, but they will result in either unappealingly large spheres on the one extreme or poorly defined spheres resembling tapioca or mush or even a single solid mass on the other extreme. Therefore, it is most preferred to use a dry granulated SAP with a particle size distribution such that the resulting hydrated spheres have a diameter distribution of from about 2 mm to about 2 cm, and that is achieved by use of a dry granulate SAP having particle size of from about 0.5 mm to about 6 mm. Most preferred is to use a mixture of two particle size distributions of the crosslinked acrylamide/potassium acrylate copolymer (CAS No. 31212-13-2) where the two particle size ranges are from about 2 mm to about 3 mm and from about 4 mm to about 5 mm. The described diameter range of 2 mm to 2 cm for the final swelled spheres is an outcome desired mostly for aesthetic reasons, and if a granular super-absorbent copolymer is available sieved to a very tight particle size distribution, this will work as well in the present invention. There is a qualitative appeal to having a mixture of sphere sizes displayed in a jar, but this is not critical. Also, the resulting spheres should not be too large as this will ultimately compromise the evaporation of the volatiles and in the extreme case of large spheres (e.g., one or two huge spheres), the air freshener will look and function more like a solid block of gel, which is not desired and beyond the scope of the present invention. A mixture of final sizes for the fragranced hydrogel spheres also allows for air movement between spheres since they cannot uniformly pack together in a vented container for use. With the preferred overall SAP particle size of from about 0.5 mm to about 6 mm, the absorption of the fragrance microemulsion by the SAP granulate typically takes between 12 and 42 hours at room temperature. The SAP and fragrance microemulsion may of course be combined in the containers in which the air freshener product is to be merchandised, and these containers may be boxed up in corrugate for shipment even without waiting for full hydration of the spheres. Certainly by the time the product reaches any retail store for merchandising display, the hydration process would be long since completed.

For optimal clarity and esthetics, the completeness of the SAP hydration must be achieved without leaving behind unused fragrance microemulsion that will appear as extra liquid in the jar of swelled fragranced spheres. For this balance to be achieved, it is preferred to use from about 0.5% to about 3.0% by weight of the SAP granules to total weight of the final composition. If too much SAP is used in relation to the amount of fragrance microemulsion, the spheres will have an opaque core, appearing to contain a seed or nucleus inside of the relatively clear swollen hydrogel sphere. As mentioned, if too little SAP is used in relation to fragrance microemulsion, the extra fragrance solution will not be absorbed by the SAP and it will remain in the decorative jar sloshing around with the hydrogel spheres, seriously destroying the aesthetic appeal of the product. In the present method, the preferred amount of SAP to total composition is from about 0.5% to about 3.0%, more preferred is SAP from about 1.0% to about 2.0%, and most preferred is SAP from about 1.4% to about 1.8% by weight in the total composition. As explained in the formula tables below, the remainder of the finished air freshener composition is the fragrance microemulsion. Thus, if the most preferred level of 1.8% by weight SAP is used in the method, 98.2% by weight is the fragrance microemulsion. Most preferred is to use a mixture of two particle size cut of the crosslinked acrylamide/potassium acrylate copolymer (CAS No. 31212-13-2) where the two particle sizes range from about 2 mm to about 3 mm and from about 4 mm to about 5 mm, and to use from about 0.5% to about 3% by weight of this coarse granulate with from about 97% to about 99.5% of fragrance microemulsion, discussed below.

Fragrance Microemulsions

The formation of a stable aqueous fragrance microemulsion typically requires the proper selection of surfactants/emulsifiers, sometimes supplemented with other co-emulsifiers and/or various solvents. Stable fragrance microemulsions are described in U.S. Pat. No. 7,226,901 (Stora); U.S. Pat. No. 6,403,109 (Stora); U.S. Pat. No. 6,071,975 (Halloran); U.S. Pat. No. 5,374,614, (Behan et al); US Application Publication 2002/0143072 (Aust); and, PCT Application Publication WO2005/123028 (Piechocki, at al), each of which are incorporated herein in their entirety. A microemulsion means a single or one-phase transparent, thermodynamically stable, mixture of two or more immiscible liquids and one or more surfactants/emulsifiers. Microemulsions are generally visibly clear or transparent, because they contain structures smaller than the wavelength of visible light, which is typically around 500 nm. A microemulsion contains structures that are spontaneously self-assembling aggregates, (i.e., “thermodynamically controlled” and not “kinetically controlled”), consisting of oil and surfactant monolayers, or water and surfactant monolayers. A microemulsion may contain oil droplets dispersed in water (O/W), water droplets dispersed in oil (W/O), or a bi-continuous structure or other structure. It will be optically clear to the naked eye because the incident light is not reflected by the small droplets of the dispersed phase. In the present invention, the oil phase is the fragrance oil, which will be discussed in detail below. Herein, a relatively small amount of fragrance oil is dispersed into water with the aid of one or more surfactants/emulsifiers to produce clear O/W microemulsions.

Below we describe some of the preferred emulsifiers, stabilizers and solvents that may be used to achieve a stable microemulsion for use in the present invention. However as mentioned, the preferred emulsification system is entirely nonionic and devoid of alcohol or other volatile solvents, in order to obtain the optical beauty of the finished hydrogel spheres and to achieve the postulated VOC restrictions for gel air fresheners. The present invention may require the use of a mixture of several different emulsifiers to achieve a stable microemulsion. However, depending on the nature of the fragrance (e.g., level of citrus oils or herbal extracts), it is possible to achieve a stable microemulsion by simply using one emulsifier along with the water and fragrance. Other fragrances require tricky combinations of several emulsifiers and possibly solvents to achieve stable, clear microemulsions. The important point is that selection of emulsifiers, stabilizers, etc., is a somewhat empirical process and is without question fragrance dependent.

Nonionic Emulsifier

The nonionic emulsifier for use in the present method of production and composition may comprise at least one nonionic material including: sorbitan esters; alkoxylated sorbitan esters; C₂-C₆ glycols; glycol esters; glycerin; glyceryl esters; alkoxylated glyceryl esters; amide waxes; fatty alcohols; monoalcohol esters; polyethylene glycol, polyethylene glycol esters; polypropylene glycol, polypropylene glycol esters, fatty alcohol alkoxylates; alkyl phenol alkoxylates; alkoxylated fatty acid esters; and other nonionic materials of surfactant classification (e.g. alkanolamides, amine N-oxides, alkylpolyglycosides, etc.), and mixtures thereof. Regardless of the nature of the nonionic material(s), it is preferred to use a total amount of nonionic emulsifier in the fragrance microemulsion at from about 0.5% to about 6% by weight, or about 1 to 2 times the amount of fragrance oil to be emulsified. For example, if the amount of fragrance in the microemulsion is 3.0% by weight, then the preferred amount of nonionic material is from about 3% to about 6% by weight.

Preferred nonionic emulsifiers for use herein include the sorbitan derivatives such as the Span®, Brij®, Tween® and Atlas® products available from Croda (formerly Uniqema). These materials are sorbitan esters generally comprising a fatty acid chain, the sorbitan linkage, and optionally an alkoxylate (e.g. polyoxyethylene, also termed “PEG”, or “EO”) chain. The more preferred nonionic emulsifier for use in the present invention includes the sorbitan esters, in particular 3-80 mole ethoxylated mono-, di-, or tri-fatty acid esters of sorbitan. These materials are available under the trade name of Tween® and Atlas® from Croda and include: polyoxyethylene (2) sorbitan monolaurate (Tween® 20); polyoxyethylene (4) sorbitan monolaurate (Tween® 21); polyoxyethylene (20) sorbitan monopalmitate (Tween® 40); polyoxyethylene (20) sorbitan monostearate (Tween® 60); polyoxyethylene (4) sorbitan monostearate (Tween® 61); polyoxyethylene (20) sorbitan tristearate (Tween® 65); polyoxyethylene (5) sorbitan monooleate (Tween® 81); polyoxyethylene (20) sorbitan monooleate (Tween® 80); polyoxyethylene (20) sorbitan trioleate (Tween® 85); and, polyoxyethylene (80) sorbitan monolaurate (Atlas® G-4280), and mixtures thereof. The sorbitan esters (i.e., non-alkoxylated) are also useful, and are available under the trade name Span® from Croda. These preferred nonionic materials include sorbitan monstearate (Span® 60); and, sorbitan tristearate (Span® 65). Most preferred is to use Tween® 20, Tween® 60 and/or Tween® 80, or mixtures thereof to create the stable fragrance microemulsion at from about 0.5% to about 6% by weight to total weight of microemulsion composition.

Other preferred nonionic emulsifiers for use herein include surfactants such as ethoxylated (EO), propoxylated (PO), or mixed ethoxylated/propoxylated (EO/PO) alkylphenol ethers; EO, PO or EO/PO C₄-C₁₆ fatty alcohols; EO, PO or EO/PO mono- and di-esters of aliphatic C₄-C₁₆ carboxylic acids; EO, PO or EO/PO branched aliphatic alcohols with a main aliphatic carbon chains of C₄-C₁₆; and, EO, PO or EO/PO hydrogenated castor oils (such as the Cremophor® materials from BASF). Preferred ethoxylated aliphatic alcohols for use in the present invention are available under the trade name Tomadol® from Tomah. The most preferred ethoxylated aliphatic alcohols for use in the present invention include Tomadol® 25-12 from Tomah, which is essentially C₁₂-C₁₅ alcohol with an average 12 moles ethylene oxide, and/or Tomadol® 91-8 from Tomah, which is essentially C₉-C₁₁ alcohol with an average 9 moles ethylene oxide. The most preferred ethoxylated hydrogenated castor oil is Cremophor® RH-40, which is PEG-40-hydrogenated castor oil. Also preferred is Eumulgin® HPS from Cognis, which is a mixture of ethoxylated alcohols, EO/PO glycol ethers, and ethoxylated hydrogenated castor oil, along with the Genapol® products from Clariant. Most preferred are combinations of these ethoxylated materials fine tuned to accommodate the fragrance type to be emulsified.

Other preferred nonionic surfactants include the amine oxide surfactants. The preferred amine oxide surfactant for use in the present invention is typically a trialkyl amine N-oxide, most preferably an alkyldimethylamine N-oxide. Examples of such materials that find use in the fragrance microemulsion herein include Ammonyx® LO from Stepan, Barlox® 12 from Lonza Corporation, and Surfox® LO Special from Surfactants, Inc. These compounds are essentially aqueous or water/alcohol solutions of lauryl- or myristyl-dimethylamine oxide or blends/chain length distributions thereof. Any of these nonionic materials or mixtures thereof may be incorporated into the fragrance microemulsion at from about 0.5% to about 6% by weight.

Other preferred nonionic materials for use in the present method to produce fragranced hydrogel spheres include the amide type nonionic surfactants, for example alkanolamides that are condensates of fatty acids with alkanolamines such as monoethanolamine (MEA), diethanolamine (DEA) and monoisopropanolamine (MIPA. Useful alkanolamides to assist in constructing a stable fragrance microemulsion for use herein include ethanolamides and/or isopropanolamides such as monoethanolamides, diethanolamides and isopropanolamides in which the fatty acid acyl radical typically contains from 8 to 18 carbon atoms. Especially satisfactory are mono- and diethanolamides such as those derived from coconut oil mixed fatty acids or special fractions containing, for instance, predominately C₁₂ to C₁₄ fatty acids. Of particular use in this method of production are mono- and diethanolamides derived from coconut oil mixed fatty acids, (predominately C₁₂ to C₁₄ fatty acids), such as those available from McIntyre Group Limited under the brand name Mackamide®. Most preferred is Mackamide® CMA, which is coconut monoethanolamide available from McIntyre. Amide surfactants, when used as the nonionic emulsifier or as a co-emulsifier in a mixture of emulsifiers, are incorporated into the fragrance microemulsion at from about 0.5% to about 6% by weight.

The fragrance microemulsion to be admixed with the SAP in the present invention may also be stabilized with alkyl polyglycoside surfactant as a nonionic material. The alkyl polyglycosides (APGs) also called alkyl polyglucosides if the saccharide moiety is glucose, are naturally derived nonionic surfactants. The alkyl polyglycosides that may be used in the present invention are fatty ester derivatives of saccharides or polysaccharides that are formed when a carbohydrate is reacted under acidic condition with a fatty alcohol through condensation polymerization. The APGs are typically derived from corn-based carbohydrates and fatty alcohols from natural oils in animals, coconuts and palm kernels. The alkyl polyglycosides that are preferred for use in the present invention contain a hydrophilic group derived from carbohydrates and is composed of one or more anhydroglucose units. Each of the glucose units can have two ether oxygen atoms and three hydroxyl groups, along with a terminal hydroxyl group, which together impart water solubility to the glycoside. The presence of the alkyl carbon chain leads to the hydrophobic tail to the molecule. When carbohydrate molecules react with fatty alcohol compounds, alkyl polyglycoside molecules are formed having single or multiple anhydroglucose units, which are termed monoglycosides and polyglycosides, respectively. The final alkyl polyglycoside product typically has a distribution of varying concentration of glucose units (or degree of polymerization). The APGs that may be used in the fragrance microemulsion as the nonionic emulsifier component preferably comprise saccharide or polysaccharide groups (i.e., mono-, di-, tri-, etc. saccharides) of hexose or pentose, and a fatty aliphatic group having 6 to 20 carbon atoms. Preferred alkyl polyglycosides that can be used according to the present invention are represented by the general formula, Gₓ-O—R¹, wherein G is a moiety derived from reducing saccharide containing 5 or 6 carbon atoms, e.g., pentose or hexose; R¹ is fatty alkyl group containing 6 to 20 carbon atoms; and x is the degree of polymerization of the polyglycoside, representing the number of monosaccharide repeating units in the polyglycoside. Generally, x is an integer on the basis of individual molecules, but because there are statistical variations in the manufacturing process for APGs, x may be a non-integer on an average basis when referred to APG used as an emulsifier for the fragrance microemulsion of the present invention. For the APGs of use herein, x preferably has a value of less than 2.5, and more preferably is between 1 and 2. Exemplary saccharides from which G can be derived are glucose, fructose, mannose, galactose, talose, gulose, allose, altrose, idose, arabinose, xylose, lyxose and ribose. Because of the ready availability of glucose, glucose is preferred in polyglycosides. The fatty alkyl group is preferably saturated, although unsaturated fatty chains may be used. Generally, the commercially available polyglycosides have C₈ to C₁₆ alkyl chains and an average degree of polymerization of from 1.4 to 1.6. APG surfactants, when used as the nonionic emulsifier or as a co-emulsifier in a mixture of nonionic materials, may be incorporated into the fragrance microemulsion at from about 0.5% to about 6% by weight.

The fragrance microemulsion herein may also be stabilized with polyether materials, such as a polyethylene or polypropylene glycol, or mixtures of these as the nonionic emulsifier. One such polyether useful in the fragrance microemulsion is polyethylene glycol (or “PEG”). These materials are most readily obtained from the Dow Chemical Company under the brand name Carbowax®. Esters of PEG may also find use in the present invention. Non-limiting examples include: PEG (40) stearate; PEG (200) cocoate; PEG (200) monooleate; PEG (300) monooleate; PEG (300) monostearate; PEG (400) cocoate; PEG (400) dilaurate; PEG (400) diooleate; PEG (400) monolaurate; PEG (400) monooleate; PEG (400) monostearate; PEG (400) ricinoleate; PEG (600) dioleate; and, PEG (600) monolaurate. The fragrance microemulsion may also utilize small molecular weigh glycols (i.e. C₂-C₆) such as ethylene glycol, propylene glycol, diethylene glycol or dipropylene glycol. Additionally, esters of these lower molecular weight glycols find use in the present invention. Some non-limiting examples include: diethylene glycol distearate; diethylene glycol monostearate; ethylene glycol monostearate; propylene glycol dioleate; propylene glycol monostearate; and, propylene glycol tricapryl caprate. Any of these glycols, glycol ethers, polyethers, and/or esters, when used as the nonionic emulsifier or as a co-emulsifier in a mixture of nonionic materials, may be incorporated into the fragrance microemulsion at from about 0.5% to about 6% by weight.

Additionally, monoalcohol esters find use in the present invention to emulsify the fragrance into a stable O/W fragrance microemulsion. These materials include: 2-ethylhexyl oleate; 2-ethylhexyl palmitate; 2-ethylhexyl tallowate; 2-ethylhexyl stearate; butyl oleate; butyl stearate; cetyl palmitate; cetyl stearate; decyl oleate; isocetyl isostearate; isocetyl stearate; isopropyl myristate; isopropyl oleate; isopropyl palmitate; isopropyl palmitate-stearate; isotridecyl stearate; isodecyl stearate; myristyl myristate; and, octyl palmitate. These alcohol esters, when used as the nonionic emulsifier or as a co-emulsifier in a mixture of nonionic materials, may be incorporated into the fragrance microemulsion at from about 0.5% to about 6% by weight.

Lastly, glycerin, glyceryl fatty acid mono-, di-, and tri-esters, and alkoxylated fatty acid glyceryl mono-esters may be used as the nonionic emulsifier herein, either alone or mixed with other nonionic materials discussed. These well known emulsifiers include such compounds as: glyceryl monostearate, monooleate, monopalmitate, monococoate, monotallowate, monomyristate, monoricinolate and the like; polyoxyethylene-glyceryl monostearate, monooleate, monopalmitate, monococoate, monotallowate, monomyristate, monoricinoleate, and the like, where the degree of ethoxylation is from about 7 to about 80; glyceryl di-stearate, -oleate, -palmitate, -cocoate, -tallowate, -myristate, -ricinolate, and the like; and, glyceryl tri-acetate, -stearate, -oleate, -palmitate, -cocoate, -tallowate, -myristate, -ricinolate, and the like. Glycerin and these glycerin derivatives, when used as the nonionic emulsifier or as a co-emulsifier in a mixture of nonionic materials, may be incorporated into the fragrance microemulsion at from about 0.5% to about 6% by weight.

It should be noted that depending on molecular weight and structure, some of these nonionic materials may be solid, waxy solid or slush at room temperature. In that case, the nonionic material may be warmed in order to liquefy it before it is premixed with the fragrance oil and then added into rapidly agitated water.

The most preferred emulsifiers, and combinations that have worked for several fragrance types ranging from citrus to herbal to “cool watery,” is a mixture of Tomadol® 25-12, lauryl dimethyl amine N-oxide, and Cremophor RH-40, at a total emulsifier level of from about 0.5% to about 6% by weight, or a mixture of Tomadol® 25-12, Tomadol® 91-8, and lauryl dimethyl amine N-oxide, at a total emulsifier level of from about 0.5% to about 6% by weight in the fragrance microemulsion composition. The latter of these two combinations has the advantage of working across a broader range of fragrances without the use of ethanol as a co-solvent, and has consistently resulted in final fragranced hydrogel spheres of superior optical clarity. The former of the two mixtures above tends to require some ethanol to achieve stable microemulsions depending on fragrance type.

Although not preferred because of the likely VOC restrictions for gel air fresheners, the present method may also include one or more solvents that may be used if the stable fragrance microemulsion is otherwise not achieved with only nonionic emulsifiers. Useful solvents for a stable fragrance microemulsion include ethanol, isopropanol, n-propanol, n-butanol, MP-Diol (methylpropanediol), ethylene glycol, propylene glycol, and other small molecular weight alkanols, diols, and polyols that may assist in emulsifying the fragrance into the water and stabilizing the emulsion when used at a level of from about 0.2% to about 7%. If the stable microemulsion is not obtained from use of nonionic emulsifiers in any combination, using the same weight percent of ethanol as fragrance oil will frequently create a clear microemulsion. For example, if 0.5% to 6% by weight of any combination of nonionic materials is still ineffective to emulsify 3% by weight fragrance oil into water, then further incorporation of 3% by weight ethanol may give the stable microemulsion, albeit one that contains unwanted VOC from the ethanol.

Fragrance Oil

The fragrance oil used in the fragrance microemulsion to be admixed with the SAP granulate may comprise one of more volatile organic substances with sensory effects available from any of the now known, or hereafter established, perfumery suppliers, such as International Flavors and Fragrances (IFF) of New Jersey, Givaudan of New Jersey, Firmenich of New Jersey, etc. Many types of fragrances can be used in the present invention. Preferably the fragrance materials are volatile essential oils. The fragrances, however, may be synthetically derived materials (aldehydes, ketones, esters, etc.), naturally derived oils, or mixtures thereof. Naturally derived fragrance substances include, but are not limited to, musk, civet, ambergis, castoreum and like animal perfumes; abies oil, ajowan oil, almond oil, ambrette seed absolute, angelic root oil, anise oil, basil oil, bay oil, benzoin resinoid, bergamot oil, birch oil, bois de rose oil, broom abs., cajeput oil, cananga oil, capsicum oil, caraway oil, cardamon oil, carrot seed oil, cassia oil, cedar leaf, cedarwood oil, celery seed oil, cinnamon bark oil, citronella oil, clary sage oil, clove oil, cognac oil, coriander oil, cubeb oil, cumin oil, camphor oil, dill oil, estragon oil, eucalyptus oil, fennel sweet oil, galbanum res., garlic oil, geranium oil, ginger oil, grapefruit oil, hop oil, hyacinth abs., jasmin abs., juniper berry oil, labdanum res., lavander oil, laurel leaf oil, lavender oil, lemon oil, lemongrass oil, lime oil, lovage oil, mace oil, mandarin oil, mimosa abs., myrrh abs., mustard oil, narcissus abs., neroli bigarade oil, nutmeg oil, oakmoss abs., olibanum res., onion oil, opoponax res., orange oil, orange flower oil, origanum, orris concrete, pepper oil, peppermint oil, peru balsam, petitgrain oil, pine needle oil, rose abs., rose oil, rosemary oil, sandalwood oil, sage oil, spearmint oil, styrax oil, thyme oil, tolu balsam, tonka beans abs., tuberose abs., turpentine oil, vanilla beans abs., vetiver oil, violet leaf abs., ylang ylang oil and like vegetable oils, etc. Synthetic fragrance materials include but are not limited to pinene, limonene and like hydrocarbons; 3,3,5-trimethylcyclohexanol, linalool, geraniol, nerol, citronellol, menthol, borneol, borneyl methoxy cyclohexanol, benzyl alcohol, anise alcohol, cinnamyl alcohol, β-phenyl ethyl alcohol, cis-3-hexenol, terpineol and like alcohols; anethole, musk xylol, isoeugenol, methyl eugenol and like phenols; α-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, citral, citronellal, hydroxycitronellal, benzaldehyde, methyl nonyl acetaldehyde, cinnamic aldehyde, dodecanol, α-hyxylcinnamic aldehyde, undecenal, heliotropin, vanillin, ethyl vanillin and like aldehydes; methyl amyl ketone, methyl β-naphthyl ketone, methyl nonyl ketone, musk ketone, diacetyl, acetyl propionyl, acetyl butyryl, carvone, menthone, camphor, acetophenone, p-methyl acetophenone, ionone, methyl ionone and like ketones; amyl butyrolactone, diphenyl oxide, methyl phenyl glycidate, gamma.-nonyl lactone, coumarin, cineole, ethyl methyl phenyl glicydate and like lactones or oxides; methyl formate, isopropyl formate, linalyl formate, ethyl acetate, octyl acetate, methyl acetate, benzyl acetate, cinnamyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, methyl salicylate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, nopyl acetate, β-phenylethyl acetate, trichloromethylphenyl carbinyl acetate, terpinyl acetate, vetiveryl acetate and like esters, and the like. Suitable fragrance mixtures may produce a number of overall fragrance type perceptions including but not limited to, fruity, musk, floral, herbaceous (including mint), watery, fresh washed linen, edible, and woody, or perceptions that are in-between (fruity-floral for example). Typically these fragrance mixtures are compounded by the fragrance houses by mixing a variety of these active fragrance materials along with various solvents to adjust cost, evaporation rates, hedonics and intensity of perception. Well known in the fragrance industry is to dilute essential fragrance oil blends (natural and/or synthetic) with solvents such as ethanol, isopropanol, hydrocarbons, acetone, glycols, glycol ethers, water, and combinations thereof. The preferred fragrance oil for use in the fragranced hydrogel spheres air freshener of the present invention may be comprised of a mixture of many fragrance actives and volatile solvents, sometimes along with smaller amounts of emulsifiers, stabilizers, wetting agents and preservatives. As is almost always the case, the compositions of the fragrance oils purchased from the various fragrance supply houses remain proprietary and thus can only be described in general terms. The fragrance oil is preferably incorporated at a level of from about 0.1% to about 10% by weight in the fragrance microemulsion, and most preferred at a level of from about 2.5% to about 3.5% depending on the fragrance type and the consumer perception of fragrance strength desired.

Optional Adjuvant

The method/composition of the present invention may also include a preservative to help prevent microbial growth in the aqueous/organic media of the hydrogel spheres. The preferred preservatives include Neolone® and Kathon® products from Lonza and Rohm & Haas. These materials are incorporated at the manufacturers' recommended levels to discourage bacterial and mold growth in the hydrogel spheres and are selected by knowing the composition and the pH of what is being preserved. Preservatives herein are meant to include anti-oxidants and uv-light absorbers. For example, butylated hydroxyl toluene (BHT) or other anti-oxidant may be added, and/or substances that absorb light and protect against dye fading (e.g. benzotriazole, benzophenone, bemotrizinol and like substances sold by BASF/Ciba under the Tinosorb® brand).

The method/composition of the present invention may also include dyes or other suitable colorants to provide aesthetic appeal to the fragranced spheres and to heighten the optical clarity and glistening from the spheres. Such dyes may include, but are not limited to, FD&C and/or D&C Yellows, Reds, Blues, Greens and Violets, or really any other dye or pigment. Most preferred dyes include FD&C Yellow #5 and Blue #1, and D&C Violet #2. Dyes are incorporated at levels sufficient to provide a light color to the spheres, for example from about 0.0001% to about 0.5% by weight in the present composition. Batches of spheres of a particular color may be combined to obtain spheres of mixed colors. There is no limit to the combinations of colors when combining spheres of different colors.

Lastly the method/composition may include pH adjustment agents. These may be incorporated into the fragrance microemulsion in order to help stabilize the emulsion and/or to change the rate of hydration of the polymer granules with the microemulsion. Such pH adjustment agents may include mineral acids and organic acids or their conjugate bases, hydroxides, carbonates, and the like. Chelants may also be incorporated to help improve the optical clarity of the resultant spheres by complexing with hardness minerals usually brought in from the water. These chelants that may find use in the present composition include tetrasodium-EDTA, NTA salts such as trisodium nitrilotriacetate, and the like. Typically, chelants for use in the present composition may be used from about 0.001% to about 1.0%. As mentioned, incorporation of any electrolyte will slow rate of hydration of the SAP granules and will usually result in opaque spheres after hydration is complete.

The Method of Production

The method for producing the fragranced hydrogel spheres of the present invention involves combining the coarse granular SAP with the stable O/W fragrance microemulsion and allowing sufficient time for formation of the discrete spheres through hydration and swelling of the SAP. As mentioned, formation of a stable aqueous fragrance microemulsion requires the emulsification of the chosen fragrance oil into water using one or more suitable nonionic surfactant/emulsifiers described above, along with any necessary co-emulsifiers and/or solvents. The fragrance microemulsion may be prepared by first mixing the emulsifiers with the fragrance oil to form a premix, and then adding that premix to the water that already has any solvent or adjuvant such as the dyes and preservatives added in. The formation of the aqueous fragrance microemulsion may be conducted at ambient temperature or at elevated temperatures. Depending on the fragrance type and the necessary combination of emulsifiers and solvents required for thermodynamic stability, the microemulsion may comprise the steps of: premixing the fragrance oil with the nonionic surfactants/emulsifiers (with any nonionic pastes or waxy solids pre-melted into liquids); dissolving dyes, preservatives and any solvent into the water; then rapidly stirring/agitating the water phase while slowing adding in the fragrance oil/nonionic premix phase. Some of this order of addition is not critical, but what is almost always required is that the fragrance and at least one of the emulsifiers are premixed and that the fragrance oil/nonionic premix be added relatively slowly (even drop-wise) to the rapidly stirred water held either at room temperature or at elevated temperature. For example, the fragrance oil may be premixed with only alcohol ethoxylate nonionic surfactants and then that premix added to rapidly agitated water that contains another emulsifier such as an alkyl dimethyl amine N-oxide surfactant. If the water or water/emulsifier, or water/solvent/emulsifier solution is warmed before the fragrance oil/nonionic premix is added, the temperature of the water or aqueous solution is preferably held at from about 25° C. to about 60° C. Any hotter than that risks flashing off volatile fragrance notes in the addition and mixing process. Agitation may be of any degree of force, from simple stirring with a paddle-blade up to high-shear mixing with a Ross homogenizer, or disperser. In all of the examples thus studied, only simple stirring was necessary when the fragrance oil premix is added to the water phase. As mentioned, adjuvant such as dyes and preservatives may already be in the water phase into which the fragrance/nonionic premix is added, or these substances can just as easily be added after the fragrance premix has already been added to the water phase. If solvent such as an alcohol or diol is necessary, it is usually premixed with the water prior to the addition of the fragrance/nonionic premix, although for some fragrance types, the co-solvent may be added to the fragrance/nonionic premix, or even to the turbid mixture resulting after the addition of the fragrance premix to the water phase. If the water was held at an elevated temperature during addition of the fragrance/nonionic premix, the common practice is to allow the final emulsion to come to room temperature to ensure that a stable, clear microemulsion resulted. As is well known in the art, stable microemulsions form when thermodynamically set to do so, and thus they may begin cloudy but can clarify over time if the kinetics of formation of the microemulsion are slow but the thermodymamics are favorable. Obviously if the oil phase has separated out from an initially turbid emulsion, the microemulsion is doomed and no length of time will likely cure it, meaning the emulsifier type(s) and/or amount(s) of emulsifier(s) were not optimized in the first place.

Once having the clear and stable fragrance microemulsion, the SAP granules are placed into a suitable container (preferably into the decorative jars the final fragrance spheres are to be merchandized in), and the liquid is poured on top. If static electricity becomes an issue, (e.g. as seen when SAP granules are placed in perfectly dry PET plastic containers), the fragrance microemulsion may be added first to the container followed by the SAP granules. Static electricity will in some instances cause the SAP granules to literally jump our from a wide-mouth container, which seriously interferes with an automated production line process of filling the containers. As an example of a merchandisable product, 4 grams of SAP granules may be added to small plastic decorative wide-mouth containers along with about 100 grams of the fragrance microemulsion. Then each container may be closed with a decorative screen, such as the one shown in USD555478 (Miiller, et. al). The decorative screen allows for the scent to be released from the hydrogel spheres as the air freshener scents the room while preventing the touching the product. The decorative screen may then be covered by a threaded closure for sealing and storage. This procedure avoids having to transfer otherwise fragile swelled hydrogel spheres from a batching vessel into these small merchandizing containers, although such a transfer of the swollen spheres could be done if necessary. The absorption process may be as slow as 12-42 hours because of the size, hardness and permeability of the SAP granules, the temperature of the absorption reaction (normally just ambient temperature in the manufacturing plant) and the level of electrolytes in the aqueous fragrance microemulsion. “Sufficient time” for the SAP granules to absorb all of the fragrance microemulsion may be as long as 2-days. Preferably the spheres are formed from the combination of from about 0.5% to about 3% hydrogel-forming SAP granules and thus from about 97% to about 99.5% fragrance microemulsion.

Compositions of the present invention are shown in Table 1. These formulas show examples of compositions that produce hydrogel spheres of various optical transparencies depending on the fragrance type, the SAP, and the particular nonionic emulsifiers used. The entries in the table are weight percent (wt. %) active material. For example, to achieve 1.0 wt. % active amine N-oxide as shown in formula 5, 3.33% of 30% active Barlox® 12 from Lonza Corporation was used. The amount of water brought into the composition from any dilute ingredient is added to the total amount of water wt. %.

| | Hydrogel Sphere Compositon (wt. % actives) | | | | |
|---|---|---|---|---|---|
| Ingredients | 1 | 2 | 3 | 4 | 5 |
| Dry granular SAP | 1.40¹ | 1.40¹ | 1.80¹ | 1.80¹ | 1.80² |
| Lauryl dimethyl amine N-oxide | 0.60 | 0.53 | 0.60 | 0.75 | 1.00 |
| Alcohol ethoxylate C₁₂-C₁₅/12EO | 3.50 | 2.00 | 3.50 | 2.75 | 2.65 |
| Alcohol ethoxylate C₉-C₁₁/8EO | — | — | — | — | 1.23 |
| Alcohol ethoxylate blend (Eumulgin HPS) | 1.00 | 0.75 | 1.00 | — | — |
| Hydrogenated castor oil/40EO (Cremophor RH-40) | — | 2.00 | 2.00 | 2.75 | — |
| Polyoxyethylene (20) Sorbitan monooleate (Tween 80) | 1.00 | — | 1.00 | — | — |
| Colorants/Preservatives | 0.01 | 0.01 | 0.01 | 0.01 | 0.01 |
| Fragrance Oil (miscellaneous notes) | 3.00 | 3.00 | 3.00 | 3.00 | 3.00 |
| Ethanol - absolute | 3.00 | 3.00 | 3.00 | 3.00 | — |
| Water | 86.49 | 87.31 | 84.09 | 85.94 | 90.31 |
| Total | 100.00 | 100.00 | 100.00 | 100.00 | 100.00 |

Dry granular SAP used above:
¹Granular polyacrylamide/sodium polyacrylate (CAS 25085-02-03)
²Granular polyacrylamide/potassium polyacrylate (CAS 31212-13-2)

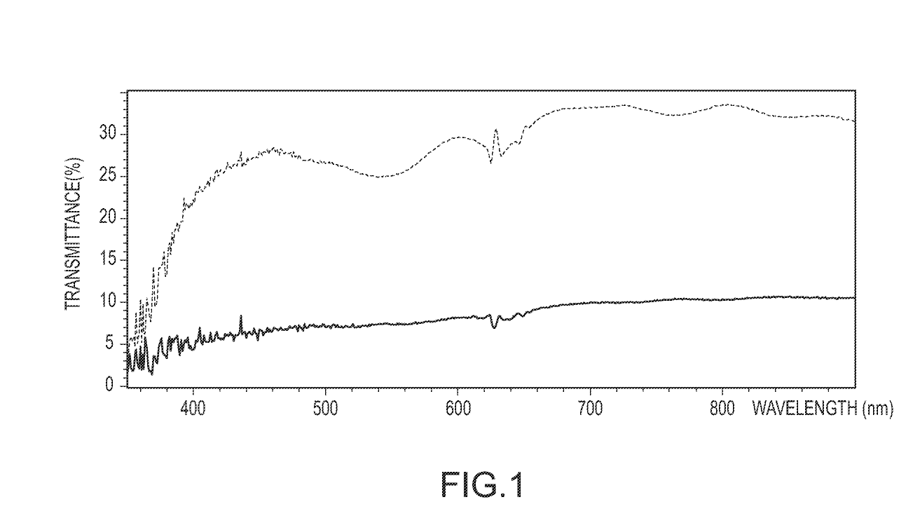

In Table 1, Formulas 1-5 use either the polyacrylamide/sodium polyacrylate SAP or the polyacrylamide/potassium polyacrylate SAP. Formula 5 gave the most visually appealing transparent spheres, whereas Formulas 1-4 produced spheres that were all somewhat cloudy in appearance. Referring now to FIG. 1, percent transmittance (% T) was measured across a spectrum of incident light from 350 nm to 900 nm in order to demonstrate the superior clarity of the spheres produced in accordance with Formula 5 in the table. The visual clarity of the hydrogel spheres was quantified using an ultraviolet-visible spectrophotometer, HP Model 8453. Measurements of percent transmittance (% T) were recorded from 350 nm to 900 nm to cover the full visible spectrum. Hydrated spheres were forced and packed into cuvettes having a pathlength of 1 cm, ensuring that contact was made throughout the cuvette by the spheres in order to have consistent incident light path lengths. The dashed line plot in FIG. 1 is the transmittance recorded for Formula 5 spheres, whereas the solid line plot in FIG. 1 is the transmittance recorded for Formula 3 spheres. Although not shown, spheres produced in accordance with Formulas 1, 2, and 4 gave transmittance plots similar to Formula 3 spheres. Consistently, the polyacrylamide/potassium polyacrylate cross-linked copolymer (CAS 31212-13-2) gave consistently clearer and more aesthetically beautiful air freshener spheres, most notably in the complete absence of ethanol solvent as exemplified by Formula 5. In the plot for Formula 5 spheres, it can be seen that the desired low-VOC spheres with optical beauty seen by the naked eye show transmittance greater than 20% at wavelengths above 400 nm, greater than 25% transmittance at wavelengths above 500 nm and greater than 30% light transmittance at wavelengths above 700 nm.

We have thus described a unique and new inventive method of production for fragranced hydrogel spheres having optical beauty that function as room air fresheners, releasing fragrance into the room environment over time. Formation of the spheres comprises the steps of first forming a stable aqueous fragrance microemulsion and then combining this liquid with super-absorbent polymer granules, most conveniently in the decorative wide-mouth container the air freshener will be sold in. The composition of the present invention will find use as an air freshener especially when the fragrance gel spheres are merchandized a decorative jar and placed in the home environment.

We claim:

1. A method for the production of low-VOC fragranced hydrogel spheres comprising the steps of:
a. preparing a stable fragrance microemulsion by combining a fragrance oil, at least one nonionic emulsifier, and water; and
b. forming discrete hydrogel spheres by combining said microemulsion with a dry granular acrylamine/acrylate super-absorbing copolymer having particle size of from about 0.5 mm to about 6 mm,
wherein said hydrogel spheres have greater than 20% light transmittance at wavelengths above 400 nm, greater than 25% light transmittance at wavelengths above 500 nm, and greater than 30% light transmittance at wavelengths above 700 nm.

2. The method of claim 1, wherein said copolymer is an acrylamide/sodium acrylate cross-linked copolymer.

3. The method of claim 1, wherein said copolymer is an acrylamide/potassium acrylate cross-linked copolymer.

4. The method of claim 1, wherein said nonionic emulsifier is selected from the group consisting of ethoxylated alcohols, propoxylated alcohols, EO/PO alcohols, amine oxides, ethoxylated hydrogenated castor oil, ethoxylated glycols, propoxylated glycols, EO/PO glycols, ethoxylated sorbitan monooleate, ethoxylated sorbitan monolaurate, ethoxylated sorbitan monopalmitate, and ethoxylated sorbitan monostearate, and mixtures thereof.

5. The method of claim 1, wherein said microemulsion further includes a solvent selected from the group consisting of alkanols, diols, and polyols, and mixtures thereof.

6. The method of claim 1, wherein said microemulsion further includes adjuvant selected from the group consisting of preservatives, colorants, pH adjusting agents, and chelants, and mixtures thereof.

7. The method of claim 4, wherein the nonionic emulsifier is selected from the group consisting of C₁₂-C₁₄/12 mole EO ethoxylated alcohol, C₉-C₁₁/9 mole EO ethoxylated alcohol, lauryl dimethylamine N-oxide, PEG-40 hydrogenated castor oil, and EO/PO glycol ether, and mixtures thereof.

8. A method for producing a low-VOC air freshener composition comprising the steps of:
a. preparing a fragrance premix by combining a fragrance oil in an amount equal to about 0.1% to about 10% by weight of the total composition with at least one nonionic emulsifier in an amount equal to about 0.5% to about 6% by weight of the total composition;
b. preparing a stable fragrance O/W microemulsion by adding said fragrance premix to agitated water, said water in an amount equal to about 85% to about 99.5% by weight of the total composition;
c. sourcing dry, coarse granular acrylamide/potassium polyacrylate cross-linked super-absorbent copolymer having granule size of from about 0.5 mm to about 6 mm; and
d. forming discrete hydrogel spheres by combining said microemulsion in an amount equal to about 97% to about 99.5% by weight of the total composition with said dry, coarse granular copolymer in an amount equal to about 0.5% to about 3% by weight of the total composition and allowing sufficient time for absorption of said water into said granules,
wherein said discrete hydrogel spheres have greater than 20% light transmittance at wavelengths above 400 nm, greater than 25% light transmittance at wavelengths above 500 nm, and greater than 30% light transmittance at wavelengths above 700 nm.

9. Discrete fragranced hydrogel spheres comprising:
a. from about 0.1% to about 10% by weight fragrance oil;
b. from about 0.5% to about 6% by weight of at least one nonionic emulsifier;
c. from about 0.5% to about 3% by weight of acrylamide/potassium polyacrylate cross-linked copolymer; and
d. from about 85% to about 99.5% by weight water,
wherein said spheres have a distribution of diameters ranging from about 2 mm to about 2 cm and have greater than 20% light transmittance at wavelengths above 400 nm, greater than 25% light transmittance at wavelengths above 500 nm, and greater than 30% light transmittance at wavelengths above 700 nm.

10. The fragranced hydrogel spheres of claim 9 further including from about 0.2% to about 7% by weight solvent selected from the group consisting of ethanol, isopropanol, n-propanol, n-butanol, methylpropanediol, ethylene glycol, and propylene glycol, and mixtures thereof.

11. The fragranced hydrogel spheres of claim 9 further including adjuvant selected from the group consisting of preservatives, colorants, pH adjusting agents, and chelants, and mixtures thereof.

12. The fragranced hydrogel spheres of claim 9, wherein the nonionic emulsifier is selected from the group consisting of C₁₂-C₁₄/12 mole EO ethoxylated alcohol, C₉-C₁₁/9 mole EO ethoxylated alcohol, lauryl dimethylamine N-oxide, PEG-40 hydrogenated castor oil, and EO/PO glycol ether, and mixtures thereof.

13. A method of scenting a room comprising the steps of:
a. placing the hydrogel spheres produced by the method of claims 1 or 8 into an open jar and placing said jar into the room to be scented.

14. A method of scenting a room comprising the steps of:
a. placing the hydrogel spheres of claim 9 into an open jar and placing said jar into the room to be scented.
